(19) United States
(12) Patent Application Publication
(10) Pub. No.: US 2022/0328193 A1
(43) Pub. Date: Oct. 13, 2022

(54) SYSTEM AND METHOD FOR ASSESSING THE RISK OF PREDIABETES

(71) Applicant: Tata Consultancy Services Limited, Mumbai (IN)

(72) Inventors: SHARMILA SHEKHAR MANDE, Pune (IN); TUNGADRI BOSE, Pune (IN); SUBHRAJIT BHAR, Pune (IN); ANIRBAN DUTTA, Pune (IN); NISHAL KUMAR PINNA, Pune (IN)

(73) Assignee: Tata Consultancy Services Limited, Mumbai (IN)

(21) Appl. No.: 17/634,903

(22) PCT Filed: Aug. 13, 2020
(86) PCT No.: PCT/IB2020/057615

§ 371 Date: Feb. 11, 2022

(30) Foreign Application Priority Data
Aug. 13, 2019 (IN) 201921032791

(51) Int. Cl. G16H 50/30; C12Q 1/6883; C12Q 1/6869

(52) U.S. Cl. CPC G16H 50/30; C12Q 1/6883; C12Q 1/6869; C12Q 2600/158

(57) ABSTRACT

Prediabetes is an intermediary physiological condition (in between healthy and diabetic states) which may be reversed through timely intervention. A system and method for assessing the risk of prediabetes in a person has been provided. The system 100 is configured to assess individuals to check the absence or presence of prediabetic symptoms, by quantifying the abundance of sensory proteins in their microbiome. The invention relates to a defined methodology that involves assessment and categorization of the person into healthy and prediabetic based on the abundance of sensory proteins in the sample collected from the faeces of the person. The systems and methods further describe microbiota based therapeutics for treatment/management of prediabetes through generating a therapeutic model and administering a consortium of healthy microbes which could modulate the disease microbiome composition towards a healthy equilibrium.

### CROSS-REFERENCE TO RELATED APPLICATIONS AND PRIORITY

The present application claims priority from Indian provisional application no. 201921032791, filed on Aug. 13, 2019. The entire contents of the aforementioned application are incorporated herein by reference.

### TECHNICAL FIELD

The embodiments herein generally relates to the field of diabetes, and, more particularly, to a method and system for assessing the risk of prediabetic condition in a person.

### BACKGROUND

Healthy pancreatic cells produce insulin, a hormone that transports sugar (glucose) to our body's tissues and maintain normal blood glucose levels. Diabetes is a condition that impairs the body's ability to process blood glucose, otherwise known as blood sugar, thereby leading to accumulation of excess glucose in blood. Diabetes could be of type 1 or type 2. Type 1 diabetes (T1D) is a chronic condition in which the pancreatic cells are destroyed by the body's immune system. Type 2 diabetes (T2D) is a chronic condition that affects the way the body processes blood sugar. In T2D, either adequate insulin is not available to maintain normal blood sugar levels, or our body tissues resist the effect of insulin owing to certain changes in the cellular receptors. While, prediabetes is the condition of a person who is predisposed to T2D. Prediabetes is an intermediary physiological condition (in between healthy and diabetic states) which may be reversed through timely intervention.

An early diagnosis of diabetes and prediabetes is important to prevent/delay added complications. Prediabetes/T2D, if not managed in a timely manner, are known to lead into other co-morbidities such as high blood pressure, obesity, abnormal cholesterol levels, cardiovascular complications, etc.

Current screening methods available for risk assessment/screening of prediabetes are not accurate enough. Method for diagnosing prediabetes involves HbA1C level of 5.7 to 6.4 percent, fasting blood glucose (FBS) level of 100 to 125, or glucose levels of 140 to 199 at two hour point of a glucose tolerance test (GTT). Notably, the blood glucose and HbA1C levels may be influenced by a large number of physiological factors (such as, haemoglobin content, survival of red blood cells, blood urea, protein uptake, alcohol consumption, stress, etc.) and are therefore inept in accurate diagnosis of prediabetes in several cases.

In addition to that, there are reported serum/biochemical/protein/urinary markers for determining a diabetic/prediabetic condition such as oral glucose tolerance test (OGTT), ketone test and micro-albumin test. However these markers show better efficiency in diagnosing diabetes and their accuracy is lacking in differentiating between healthy and prediabetic conditions. Additionally, most of them require phlebotomist expertise and/or specialized sample storage/processing facility. This, at times restricts their usage in rural/under developed settings.

Recently, a few studies have also identified the association of gut microbiome (change in composition and function) in development of Type 2 diabetes. However, no such studies could identify any microbiome based signals that could distinguish between healthy and prediabetic subjects with sufficient accuracy.

Currently, there is no well-defined medical treatment specific for prediabetes except maintaining an active lifestyle with a balanced diet which can prevent/slow the progression into Type 2 diabetes. The current treatment regime for Type 2 diabetes involves antidiabetic drugs, insulin therapy, change of lifestyle and dietary restrictions. Antidiabetic drugs are known for their adverse side effects on human health, often causing insulin resistance and further complicating the condition of the patient. In addition, insulin therapy involves routine administration of insulin injections, causing pain and trauma among patients.

### SUMMARY

Embodiments of the present disclosure present technological improvements as solutions to one or more of the above-mentioned technical problems recognized by the inventors in conventional systems. For example, in one embodiment, a system for assessing the risk of prediabetes in a person has been provided. The system comprises a sample collection module, a DNA extractor, a sequencer, a database creation module, one or more hardware processors and a memory. The sample collection module collects a microbiome sample from fecal of the person for the assessment of the risk of prediabetes, wherein the microbiome sample comprising microbial cells. The DNA extractor extracts DNA from the microbial cells. The sequencer sequences the extracted DNA to get sequenced metagenomic reads. The database creation module creates a database of sensory protein sequences of a plurality of organisms, wherein the database of sensory protein sequences comprises information pertaining to the sensory proteins of all fully sequenced bacterial genomes obtained from a plurality of public repositories. The memory in communication with the one or more hardware processors, wherein the one or more first hardware processors are configured to execute programmed instructions stored in the memory, to: generate sensory protein abundance profiles of case-control samples obtained from publicly available data; apply a random forest classifier on the generated sensory proteins abundance profiles of case-control samples to generate a classification model; quantify the abundance of a sensory protein from the sequenced metagenomic reads using the database of sensory protein sequences; assess the risk of the person to be in the prediabetes diseased state using the classification model and the quantified abundance of the sensory protein in the metagenomic sample of the person, wherein the assessment results in the categorization of the person either in a low risk or a high risk of prediabetes diseased state based on a predefined criteria; and provide a therapeutic construct to the person depending on the risk of the prediabetes.

In another aspect, a method for assessing the risk of prediabetes in a person has been provided. Initially, a database of sensory protein sequences of a plurality of organisms is created, wherein the database of sensory protein sequences comprises information pertaining to the sensory proteins of all fully or partially sequenced bacterial genomes obtained from a plurality of public repositories. Further sensory protein abundance profiles of case-control samples obtained from publicly available data is generated. In the next step, a random forest classifier is applied on the generated sensory protein abundance profiles of case-control samples to generate a classification model. Further, a microbiome sample is collected from fecal of the person for the assessment of the risk of prediabetes, wherein the microbiome sample comprising microbial cells. Further, DNA is extracted from the microbial cells. The extracted DNA is then sequenced to get sequenced metagenomic reads. Further, the abundance of a sensory protein from the sequenced metagenomic reads is quantified using the database of sensory protein sequences. Further, the risk of the person to be in the prediabetes diseased state is assessed using the classification model and the quantified abundance of the sensory protein in the metagenomic sample of the person, wherein the assessment results in the categorization of the person either in a low risk or a high risk of prediabetes diseased state based on a predefined criteria. And finally, a therapeutic construct is provided to the person depending on the risk of the prediabetes.

In yet another aspect, one or more non-transitory machine readable information storage mediums comprising one or more instructions which when executed by one or more hardware processors cause assessing the risk of prediabetes in a person. Initially, a database of sensory protein sequences of a plurality of organisms is created, wherein the database of sensory protein sequences comprises information pertaining to the sensory proteins of all fully or partially sequenced bacterial genomes obtained from a plurality of public repositories. Further sensory protein abundance profiles of case-control samples obtained from publicly available data is generated. In the next step, a random forest classifier is applied on the generated sensory protein abundance profiles of case-control samples to generate a classification model. Further, a microbiome sample is collected from fecal of the person for the assessment of the risk of prediabetes, wherein the microbiome sample comprising microbial cells. Further, DNA is extracted from the microbial cells. The extracted DNA is then sequenced to get sequenced metagenomic reads. Further, the abundance of a sensory protein from the sequenced metagenomic reads is quantified using the database of sensory protein sequences. Further, the risk of the person to be in the prediabetes diseased state is assessed using the classification model and the quantified abundance of the sensory protein in the metagenomic sample of the person, wherein the assessment results in the categorization of the person either in a low risk or a high risk of prediabetes diseased state based on a predefined criteria. And finally, a therapeutic construct is provided to the person depending on the risk of the prediabetes.

It is to be understood that both the foregoing general description and the following detailed description are exemplary and explanatory only and are not restrictive of the invention, as claimed.

### BRIEF DESCRIPTION OF THE DRAWINGS

The embodiments herein will be better understood from the following detailed description with reference to the drawings, in which:

FIG. 1 illustrates a block diagram of a system for assessing the risk of prediabetes in a person according to an embodiment of the present disclosure.

FIG. 2 shows a flowchart for creating a database of sensory protein abundances according to an embodiment of the disclosure.

FIG. 3 shows a block diagram for generating a classification model to be used in the system of FIG. 1 according to an embodiment of the disclosure.

FIG. 4A-4B is a flowchart illustrating the steps involved in assessing the risk of prediabetes in the person according to an embodiment of the present disclosure.

### DETAILED DESCRIPTION OF EMBODIMENTS

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered as exemplary only, with the true scope being indicated by the following claims.

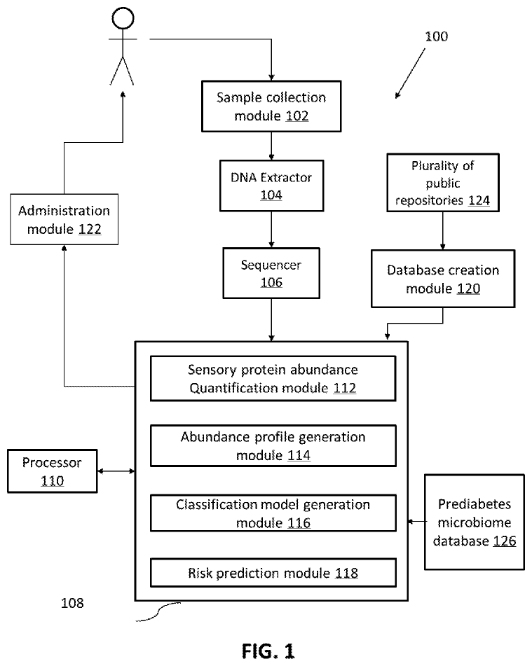

Referring now to the drawings, and more particularly to FIG. 1 through FIG. 4B, where similar reference characters denote corresponding features consistently throughout the figures, there are shown preferred embodiments and these embodiments are described in the context of the following exemplary system and/or method.

According to an embodiment of the disclosure, a system 100 for assessing the risk of prediabetes in a person is shown in FIG. 1. The system 100 is configured to assess individuals to check the absence or presence of prediabetic symptoms, by quantifying the abundance of sensory proteins in their microbiome. The invention relates to a defined methodology that involves assessment and categorization of the person into healthy and prediabetic based on the abundance of sensory proteins in the sample collected from the faeces of the person. The systems and methods further describe microbiota based therapeutics for treatment/management of prediabetes through generating a therapeutic model and administering a consortium of healthy microbes which could modulate the disease microbiome composition towards a healthy equilibrium.

According to an embodiment of the disclosure, the system 100 comprises of a sample collection module 102, a DNA extractor 104, a sequencer 106, a memory 108 and a processor 110 as shown in FIG. 1. The processor 110 is in communication with the memory 108. The processor 110 is configured to execute a plurality of algorithms stored in the memory 108. The memory 108 further includes a plurality of modules for performing various functions. The memory 108 may include a sensory protein abundance quantification module 112, an abundance profile generation module 114, a classification model generation module 116 and a risk prediction module 118. The system 100 also comprises a database creation module 120 created using a plurality of public repositories 124. The system 100 further comprises an administration module 122 as shown in the block diagram of FIG. 1. The system 100 also comprises a prediabetes microbiome database 126 as shown in the block diagram of FIG. 1.

According to an embodiment of the disclosure, the microbiome sample is collected using the sample collection module 102. The sample collection module 102 is configured to collect gut microbiome sample from a faecal sample of a subject. The microbiome sample in the form of saliva/stool/blood/other body fluids/swabs can also be collected from at least one body site/locations other than the gut e.g. oral, skin, lung etc. The microbiome sample can also be collected from subjects of different geographies. The sample can also be collected from the person from one or multiple body sites at various stages before and after successful assessment of prediabetes. Moreover, the samples can also be collected from other mammals such as cow, dog, etc. The sample collection module 102 can include a variety of software and hardware interfaces, for example, a web interface, a graphical user interface, and the like and can facilitate multiple communications within a wide variety of networks N/W and protocol types, including wired networks, for example, LAN, cable, etc., and wireless networks, such as WLAN, cellular, or satellite.

The system 100 further comprises the DNA extractor 104 and the sequencer 106. DNA is first extracted from the microbial cells constituting the microbiome sample using laboratory standardized protocols by employing the DNA extractor 104. Next, sequencing is performed using the sequencer 106 to obtain the sequenced metagenomic reads. The sequencer 106 performs whole genome shotgun (WGS) sequencing from the extracted microbial DNA, using a sequencing platform after performing suitable pre-processing steps (such as, sheering of samples, centrifugation, DNA separation, DNA fragmentation, DNA extraction and amplification, etc.) The extracted and sequenced DNA sequences are then provided to the processor 110.

In another embodiment of the disclosure, the DNA extractor 104 and sequencer 106 are also configured to use universal primers to kinase domains to specifically pull down and amplify DNA sequences fragments encoding for sensory kinases. Other embodiments can also perform amplicon sequencing (such as, sequencing 16S rRNA gene, sequencing cpn60 gene, etc.) of the collected microbiome. Further, the DNA extractor 104 and the sequencer 106 are also configured to extract and sequence microbial transcriptomic (also referred to as meta-transcriptomic) data. The DNA extractor 104 and the sequencer 106 are also configured to perform any one of chip based hybridization, ELISA based separation, size/charge based seclusion of specific class of DNA/RNA/protein and subsequently performs amplification and sequencing and/or quantification of the same. Sequencing may be performed using approaches which involve either a fragment library or a mate-pair library or a paired-end library or a combination of the same. Sequencing may also be performed using any other approaches such as by recording changes in the electric current while passing a DNA/RNA molecule through a nano-pore while applying a constant electric field or by using mass spectrometric techniques.

According to an embodiment of the disclosure, the system 100 comprises the database creation module 120. The database creation module 120 is configured to create a database of sensory protein sequences of all the organisms, wherein the database of sensory protein sequences comprises information pertaining to the proteins of all fully sequenced bacteria obtained from a plurality of public repositories 124. The plurality of public repositories may include, but not limited to NCBI, Protein Data Bank, KEGG, PFAM, EggNOG, etc. Thus, the database creation is a onetime process. The pre-created database of sensory protein sequences can be used for the diagnosis of prediabetes as explained in the later part of the disclosure.

In another embodiment of the disclosure, the database of sensory proteins created using the database creation module 120 may also include sensory protein sequences from partially sequenced bacterial genomes and/or genomes of other microorganisms including but not restricted to viruses, fungi, micro-eukaryotes, etc.

According to an embodiment of the disclosure, the memory 108 comprises the sensory protein abundance quantification module 112. The sensory protein abundance quantification module 112 is configured to compute the abundance of the sensory protein encoding genes in the sequenced metagenomic reads using the database of sensory protein sequences. In an embodiment, following methodology can be used to compute the sensory protein abundance for the sequenced metagenomic reads.

Step 1: Perform a sequence alignment such as tBLASTN with the sequences in the created sensory protein sequence database as query against the sequenced metagenomic reads. The hits satisfying a minimum e-value threshold of 1.0*e⁻⁵ (0.00001) were considered as correct matches.

Step 2: For each bacterial strain in the sensory protein sequence database the cumulative of the matches of the sequenced metagenomic reads are computed to form the “Count of sensors” which indicates approximately the potential number of sensory protein coding regions in the genome for that particular bacterial strain for the microbiome sample from which the sequenced metagenomic reads were obtained. Also for each bacterial strain in the sensory protein sequence database the cumulative length of the nucleotide bases for all these hits is computed to form the “Covered base length” which indicates approximately the total length of the potential sensory protein coding regions in the genome for that particular bacterial strain for the microbiome sample from which the sequenced metagenomic reads were obtained.

Step 3: The calculation of the sensory protein abundance can be performed using two implementations: In the first implementation, computation of sensory protein abundance is performed by calculation of the ratio of the “Count of sensors” to the total size of the sequenced metagenomic reads constituting the microbiome sample, henceforth referred to as metagenomic size (in Megabases). This ratio indicates the cumulative number of sensory proteins for that bacterial strain coded per unit of the sequenced metagenomic reads constituting the microbiome sample. Thus,

$$Sensory protein abundance = \frac{Count of Sensors for a particular strain}{Metagenomic Size}$$

In the second implementation, computation for the sensory protein abundance can be performed by calculation of the ratio of the “Covered base length” to the total metagenomic size (in Megabases) of the microbiome sample for each available bacterial strain. This ratio indicates the cumulative length of sensory protein coding regions (coding sequence) for that bacterial strain per unit of the sequenced metagenomic reads constituting the microbiome sample. Thus,

$$Sensory protein abundance = \frac{Covered base length for a particular strain}{Metagenomic Size}$$

The sensory protein abundance for the sequenced metagenomic reads can also be computed using various other implementations of the process and are described as follows. In one implementation, the computation can be performed at any of the known taxonomic levels or the computation can also be performed at each of the different taxonomic levels using a mixture of organisms. The sensory protein abundance is initially computed for each available strain(s) and in one implementation can be cumulated to a desired taxonomic level. In other implementations, the computed sensory protein abundance may be replaced by any other statistical means, such as mean, median, mode, etc. Organisms other than bacteria (either alone or in combination with other taxonomic lineages) may also be employed. In yet another implementation, one or more group of proteins, other than sensory proteins may be used, either alone or in combination with the sensory proteins and/or taxonomic classifications.

According to an embodiment of the disclosure, the memory 108 also comprises the abundance profile generation module 114, the classification model generation module 116 and the risk prediction module 118. The abundance profile generation module 114 is configured to generating abundance profiles from sequenced metagenomic reads obtained from publicly available data. The set of sequenced metagenomic reads can be used for training and/or testing. The abundance profiles of the sequenced metagenomic reads is used as the training and/or testing data for the generation of a model and testing its efficiency.

The classification model generation module 116 is configured to apply a random forest (RF) classifier on the abundance profiles of the subset of sequenced metagenomic reads to generate a classification model and test prediction accuracy on the other subset. In one embodiment, the microbiome samples, constituting of sequenced microbiome reads may be obtained from publicly available prediabetes microbiome data through prediabetes microbiome database 126. The microbiome samples, from which the sequenced metagenomic reads are obtained, are divided in a random set of 90% as the training set and rest of the 10% as the testing set. Thus, the generated classification model can also be used to classify the testing set as well.

The risk prediction module 118 is configured to assess the presence of prediabetes from the microbiome of the person providing fecal sample for risk assessment using the classification model, wherein the assessment results in the categorization of the person either in a low risk or a high risk of prediabetes based on predefined criteria. The machine learning technique of RF classifier was used for model based prediction using train and test set.

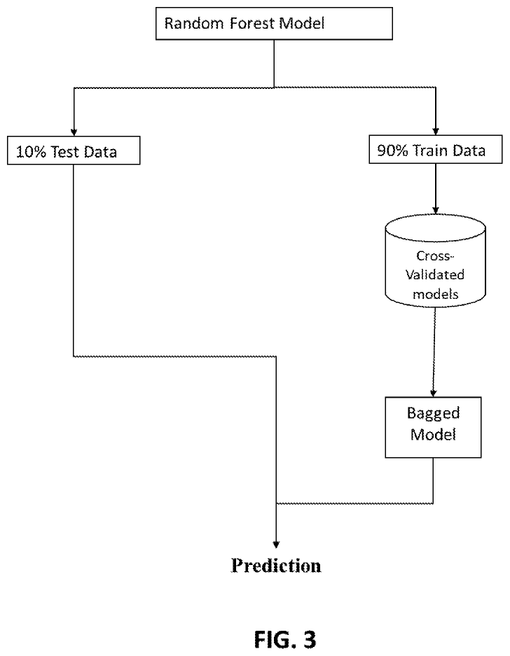

The classification model generation module 116 further creates a binary classification model as shown in FIG. 3. The binary classification model computes the risk of prediabetes using the machine learning technique of model based prediction by means of the Random Forest algorithm. Random forest approach (R 3.0.2, randomForest4.6-7 package) was applied on the sensory protein abundance profiles of case-control sequenced microbiome reads which constituted the microbiome samples. A random set of 90% of the sequenced microbiome reads which constituted the microbiome samples were selected as the training set and rest of the 10% were considered as the test set.

The current implementation was computed using species level sensory protein abundance. The alternate implementations are:
Using Abundance values of Sensory proteins (or any other group of proteins) at other taxonomic levels
Using the Sensory (or any other group of proteins) count instead of covered base length
Using any other model based prediction algorithm

According to an embodiment of the disclosure, the system 100 also comprises of the administration module 122. The administration module 122 is configured to provide/administer a therapeutic construct to the person depending on the risk of the prediabetes. It should be appreciated that any of the well-known technique can be used to administer the construct. The administration module 122 uses at least one of a consortium/construct of healthy microbes, antibiotic drugs and pre/pro-/syn-/post-biotics and fecal microbiome transplant that would help the patient's gut microbiome to attain a healthy equilibrium without any adverse health effects. The therapy may be provided in the form of any one (or a combination) of the known routes of administrations like intravenous solution, sprays, Band-Aids, pills, syrup, mouth wash etc.

The therapeutics is suggested as a consortium of microbes based on their (inverse) correlation with the disease microbiome which can contribute to the therapeutic treatment for prediabetes by modulating the disease microbiome towards healthy equilibrium. Different implementations to identify the suitable therapeutic candidates are as following:
The sub-set of the reported screening markers abundant in healthy subjects, i.e. Healthy Therapeutic Markers (HTMs) which have been previously identified in research to be non-pathogenic
The different species and strains belonging to the same genus of the HTMs which have been previously identified in research to be non-pathogenic
All organisms having >90% identity and coverage over the genome of HTMs and which have been previously identified in research to be non-pathogenic
Any previously reported organisms which are known to boost the population of (non-pathogenic) HTMs and which have been previously identified in research to be non-toxic and do not cause any adverse effect
One or more of a natural or synthetically derived compounds which boost the population of (non-pathogenic) HTMs, wherein the natural or synthetically derived compounds are non-toxic
Any organism with identical sensory protein/kinase domain to HTMs and previously identified in research to be non-pathogenic/non-toxic
one or more of a natural or synthetically derived compounds which targets the reported screening markers abundant in diseased subjects, i.e. Disease Markers (DMs), wherein the natural or synthetically derived compounds are non-toxic and do not cause any adverse effect
Any organism previously reported, or any of its related similar organisms (similar through genomic make up or characteristic functions) which inhibit growth of reported screening markers abundant in diseased patients, i.e. Disease markers (DMs) and previously identified in research to be non-pathogenic.
Any sequence with above mentioned similarity to these sequences are also potential markers.

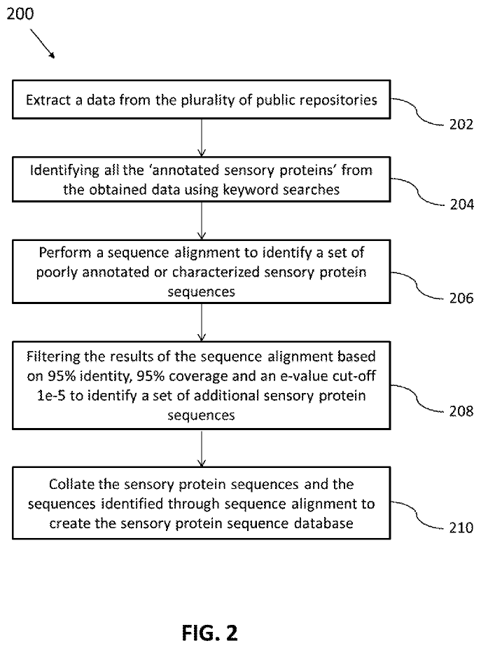

A flowchart 200 for creating a database of sensory protein sequence is shown in FIG. 2. Initially at step 202, a data is extracted from the plurality of public repositories 124. In the next step 204, all the ‘annotated sensory proteins’ from the obtained data were identified using keyword searches. At step 206, followed by a sequence alignment step (BLAST) to identify the poorly annotated/less characterized sensory protein sequences. For the purpose, the sequences corresponding to the ‘annotated sensory proteins’ were used as the database and the rest of the obtained bacterial protein sequences were used as query. At step 208, the results of the sequence alignment is filtered based on 95% identity, 95% coverage and an e-value cut-off 1.0*e⁻⁵ (0.00001) to identify a set of additional sensory protein sequences;

And finally at step 210, the sensory protein sequences (those used as a database for the BLAST search) and the ones identified through Basic Local Alignment Search Tool (BLAST) analysis were collated into the sensory protein sequence database. In another embodiment, the database creation module 120 is also configured to create the database of interactome proteins and create a database of any other types of protein group/functional class.

In another embodiment of the disclosure, the sequence alignment may be performed using other techniques such as BLAT, DIAMOND alignment tool, RAPSearch tool, Burrows-Wheeler aligner (BWA), Bowtie or through the use of clustering algorithms like BLASTCLUST, CLUSTALW, vsearch or any other heuristic techniques of identifying sequence/motif similarity.

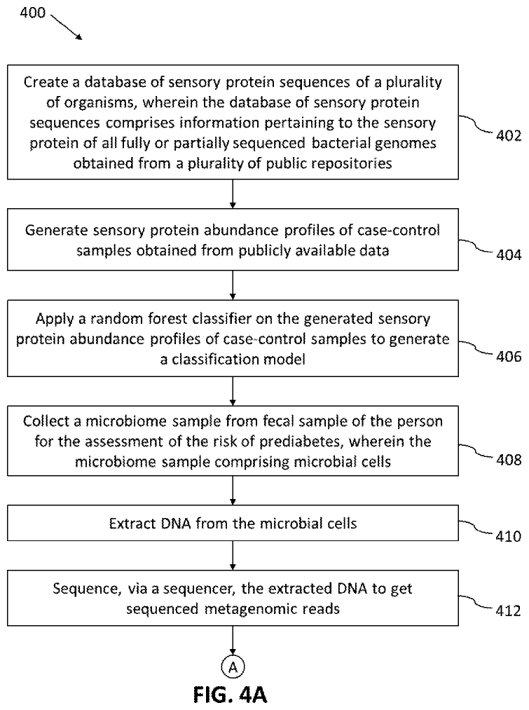

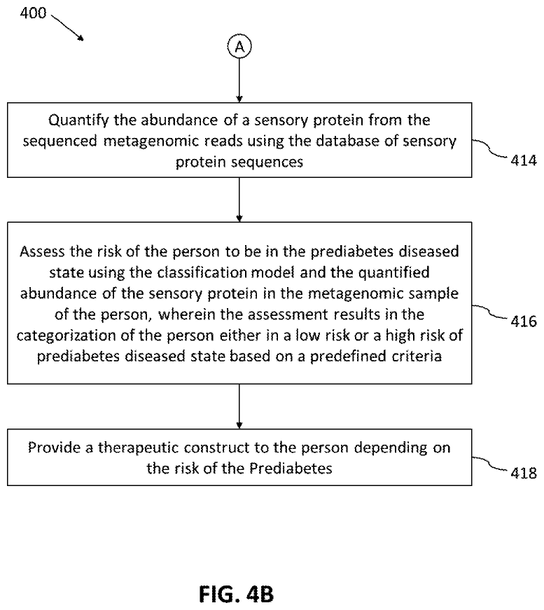

In operation, a flowchart 400 illustrating the steps involved for assessing the risk of prediabetes is shown in flowchart of FIG. 4A-4B. Initially at 402, a database of sensory protein sequences of a plurality of organisms is created, wherein the database of sensory protein sequences comprises information pertaining to the proteins of all fully sequenced bacteria obtained from a plurality of public repositories. The database of sensory protein sequences created through database creation module 120 comprises information pertaining to the proteins of all fully or partially sequenced bacteria obtained from a plurality of public repositories 124. It may be appreciated that the database creation is a one-time process and created before the test sample from a person/patient is provided for the diagnosis and thereafter therapeutic purposes. Further at step 404, the abundance profiles of case-control samples obtained from publicly available data is generated. At step 406, a random forest classifier is applied on the generated sensory protein abundance profiles of case-control samples to generate a classification model using the classification model generation module 116. It may be appreciated that this generation of the classification model is a one-time process and created before the test sample from a person/patient is provided for the diagnosis and thereafter therapeutic purposes.

In the next step 408, a microbiome sample from fecal of the person is collected for the assessment of the risk of prediabetes, wherein the microbiome sample comprising microbial cells. Later at step 410, DNA is extracted from the microbial cells using DNA extractor module 104. At step 412, the extracted DNA is sequenced via the sequencer 106, to get sequenced metagenomic reads. In the next step 414, the abundance of a sensory protein is quantified from the sequenced metagenomic reads using the database of sensory protein sequences. At step 416, the risk of the person to be in the prediabetes diseased state is assessed using the classification model and the quantified abundance of the sensory protein in the metagenomic sample of the person, wherein the assessment results in the categorization of the person either in a low risk or a high risk of prediabetes diseased state based on a predefined criteria. It may be appreciated that this generation of the prediabetes classification model is a onetime process and created before the test microbiome sample from a person/patient is provided for the diagnosis and thereafter therapeutic purposes, using publicly available data. And finally at step 418, a therapeutic construct is provided to the person depending on the risk of the prediabetes.

According to an embodiment of the disclosure, the system 100 for assessing the risk of prediabetes in the person can also be explained with the help of following example. Publicly available gut microbiome data in the form of stool/faecal microbiome samples obtained from a previously published study was used for this evaluation. In this study, the number of faecal samples corresponding to prediabetic condition and controls were taken. The sequenced metagenomic reads obtained from 91 metagenomic shotgun-sequenced faecal microbiome samples were used in the current evaluation and analysis.

In this implementation, DNA fragments encoding for the set of kinase proteins which have been identified to be key differentiators between healthy and prediabetic samples may be specifically measured using a PCR-based approach (such as, rtPCR, qPCR, etc.) or ELISA-based technique. In this case, primers specific to the proteins of interest may be designed to pull down the proteins of interest. This would enable for designing a prediabetes test kit which is highly affordable and can be used assessment of prediabetes risk among masses.

A pairwise alignment using tBLASTN, was performed using the derived sensory protein sequence database as query against the sequenced metagenomic reads. The protein-nucleotide translated BLAST or tBLASTN performs a comparison of a protein type query against all 6-frame translations of a nucleotide database. The blast hits satisfying the e-value threshold of 1.0*e⁻⁵ (0.00001) were used to calculate the sensory protein abundance across all bacterial strains, which constituted the sensory protein sequence database. For the current implementation the sensory protein abundance were calculated at species level. Sensory protein abundance was computed by cumulating the abundance of sensory proteins for all the bacterial strains, constituting the sensory protein sequence database, of a particular species for each of the stool/faecal microbiome samples.

State of the art machine learning technique was implemented for model based prediction of the samples. Random forest (R 3.0.2, random Forest 4.6-7 package) was applied and a random set of sequenced metagenomic reads comprising 90% of the microbiome samples were selected as the training set and rest of the 10% were considered as the test set. Subsequently 10 replicates on 10-fold cross-validation were performed on the train dataset to build 100 cross-validation RF models. Furthermore the ‘importance’ of each of the features included in the cross-validation models was captured in form of GINI importance. ‘X’ most ‘important’ features (here X was equal to 10), based on GINI importance values were selected from each of the 100 models (in alternate implementations, X may vary from 2 to ‘N’, wherein ‘N’ is the total number of features). Each feature in the sub-set of features, that was obtained by choosing the ‘X’ most ‘important’ features from each of the 100 cross-validation RF models, was subsequently ranked on the basis of the sum of their GINI importance values (in alternate implementation, the features may be ranked on the basis of their occurrence frequency in the sub-set of features). Next, multiple ‘evaluation’ models were obtained by cumulatively adding the next ranked feature in the feature sub-set with the features of the previous ‘evaluation’ model, wherein the first ‘evaluation’ model comprised of the top two features in the feature sub-set. Subsequently, the performance of all the ‘evaluation’ models were assessed on the basis of their performance and the best performing ‘evaluation’ model was chosen as the final ‘bagged’ model. The performance of the ‘evaluation’ models was evaluated on the basis of Balancing Score, followed by MCC and AUC scores. In cases where multiple models demonstrated identical performance measures, the ‘evaluation’ model with least number of features was chosen as the final ‘bagged’ model. The Balancing Score was computed as following.

Balancing Score=(sensitivity+specificity)−absolute (sensitivity−specificity)

The final ‘bagged’ model was then validated on the test set containing rest 10% of the dataset earlier kept aside as the independent test set. The accuracy of training model and the confidence probability of the binary prediction to be ‘case’ or ‘control’ (prediabetic or healthy) were accounted. Table I below shows the cross validation results of the study:

Cross-Validation Results

TABLE I

| Classification | Train | | Test | |
|---|---|---|---|---|
| Basis | Sensitivity | Specificity | Sensitivity | Specificity |
| Taxonomy | 72.09 | 64.10 | 60.00 | 50.00 |
| (Genus) | | | | |
| Taxonomy | 74.42 | 66.67 | 60.00 | 50.00 |
| (Species) | | | | |
| Sensory | 76.74 | 74.36 | 60.00 | 75.00 |
| Proteins | | | | |
| Kinase | 76.74 | 74.36 | 60.00 | 62.50 |
| proteins* | | | | |

*Refer to results obtained using an alternate implementation wherein a subset of proteins (those containing a kinase domain) in the sensory protein database is used as the backend database. Using this subset of proteins allow for preparing a test kit and a prediabetes screening protocol that is highly economical and can be easily deployed for mass screening. Table II below shows the list of discriminating taxa (based on Sensory protein Abundance):

TABLE II

| Taxonomy | Healthy | Prediabetic |
|---|---|---|
| Acholeplasma palmae | 0.064753 | 0.1050695 |
| Haemophilus influenzae | 9.6692 | 8.100685 |
| Oceanithermus profundus | 4.23123 | 3.71662 |
| Pseudoxanthomonas spadix | 2.54792 | 2.195825 |
| Rhodanobacter denitrificans | 0.0625 | 0.03377015 |
| Rhodothermus marinus | 10.8521 | 9.567845 |
| Thermaerobacter | 4.06502 | 3.56249 |
| marianensis | | |

Based on the results presented, one or more of the non-pathogenic HTMs, viz, Oceanithermus profundus, Pseudoxanthomonas spadix, Rhodothermus marinus, Thermaerobacter marianensis or other non-pathogenic organisms satisfying one or more of the above criteria may be administered either alone or in concoction for therapeutic purposes. Further, one or more of the DMs comprising at least of Acholeplasma palmae may be targeted using antibiotics.

Thus the Random forest model based prediction method applied can efficiently perform in risk assessment of prediabetes, based on sensory protein abundance from the faecal microbiome sample. The sensory protein abundance is clearly a potential biomarker for prediction of diseased state and can be similarly employed for diagnostic purposes in case of other diseases and disorders. The disclosure provides a non-invasive and cost effective method as compared to the existing methods. The embodiments of present disclosure herein provide a method and system for assessing the risk of prediabetes in the person.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein addresses unresolved problem of early assessment of prediabetes in the person. The embodiment provides a system and method to assess the risk of prediabetes in a person. Further depending on the risk, the therapeutic construct is also provided.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

1. A method for assessing the risk of prediabetes in a person, the method comprising:
creating, via one or more hardware processors, a database of sensory protein sequences of a plurality of organisms, wherein the database of sensory protein sequences comprises information pertaining to the sensory protein of all fully or partially sequenced bacterial genomes obtained from a plurality of public repositories, wherein the creating further comprises:
extracting a data from the plurality of public repositories,
identifying all annotated sensory proteins from the extracted data using a set of keyword searches,
performing a sequence alignment to identify a set of poorly annotated or characterized sensory protein sequences,
filtering the results of the sequence alignment based on 95% identity, 95% coverage and an e-value cut-off 1.0*e⁻⁵ (0.00001) to identify a set of additional sensory protein sequences, and
collating the sensory protein sequences and the sequences identified through sequence alignment to create the database of sensory protein sequences;
generating, via the one or more hardware processors, sensory protein abundance profiles of case-control samples obtained from publicly available data;
applying, via the one or more hardware processors, a random forest classifier on the generated sensory protein abundance profiles of case-control samples to generate a classification model, wherein the classification model generation comprises:
applying a Random Forest (RF) approach on the sensory protein abundance profiles of sequenced metagenomic reads,
selecting a random set of sequenced metagenomic reads comprising 90% of the microbiome samples as a training set and rest of the 10% were considered as a test set,
performing 10 replicates on 10-fold cross-validation on the training set to build 100 cross-validation RF models,
capturing an importance of each of the features included in cross-validation models in terms of GINI index,
selecting a predefined number of most ‘important’ features based on GINI index values from each of the 100 cross-validation RF models to obtain a feature sub-set,
ranking each of the features in the feature sub-set, on the basis of the sum of their GINI index values,
obtaining multiple evaluation models by cumulatively adding the next ranked feature in a sub-set of features with the features of the previous ‘evaluation’ model, wherein the first ‘evaluation’ model comprised of the top two features in the feature sub-set,
assessing the performance of all the ‘evaluation’ models on the basis of their added features,
choosing the best performing ‘evaluation’ model based on the assessed performance as the final classification model,
evaluating the performance of the ‘evaluation’ model on the basis of a balancing Score, followed by Matthews correlation coefficient (MCC) and Area under the curve (AUC) scores, and
validating the final classification model on the test set containing rest 10% of the dataset earlier kept aside as the independent test set, wherein the accuracy of training model and the confidence probability of the binary prediction to be ‘case’ or ‘control’ (schizophrenic or healthy) were accounted;
collecting a microbiome sample from fecal sample of the person for the assessment of the risk of prediabetes, wherein the microbiome sample comprising microbial cells;
extracting DNA from the microbial cells;
sequencing, via a sequencer, using the extracted DNA to get sequenced metagenomic reads;
quantifying, via the one or more hardware processors, the abundance of a sensory protein from the sequenced metagenomic reads using the database of sensory protein sequences;
assessing, via the one or more hardware processors, the risk of the person to be in the prediabetes diseased state using the classification model and the quantified abundance of the sensory protein in the metagenomic sample of the person, wherein the assessment results in the categorization of the person either in a low risk or a high risk of prediabetes diseased state based on a predefined criteria; and
providing a therapeutic construct to the person depending on the risk of the prediabetes.

2. The method according to claim 1, wherein the therapeutic construct comprises one or more -non-pathogenic Healthy Therapeutic Markers (HTMs) abundant in healthy population, a plurality of antibiotic drugs targeted against Disease Markers (DMs), pre-/pro-/syn-/post-biotics and fecal microbiome transplant to help the person's gut microbiome to attain a healthy equilibrium.

3. The method according to claim 1, wherein, the therapeutic construct comprises one or more of:
a plurality of Healthy Therapeutic Markers (HTMs), wherein the plurality of Healthy Therapeutic Markers is non-pathogenic,
species and strains belonging to same genus of the HTMs, wherein the species and strains are non-pathogenic,
a plurality of organisms having more than 90 percent identity and coverage over the genome of HTMs, wherein the plurality of organisms are non-pathogenic,
one or more organisms which boost the population of HTMs, wherein the one or more organisms are non-pathogenic,
one or more of a natural or synthetically derived compounds which boost the population of HTMs, wherein the natural or synthetically derived compounds are non-toxic, or
one or more of a natural or synthetically derived compounds which targets the Disease Markers (DMs), wherein the natural or synthetically derived compounds are non-toxic and do not cause any adverse effect.

4. The method according to claim 3, wherein the plurality of Healthy Therapeutic Markers (HTMs) comprises one or more of Oceanithermus profundus, Pseudoxanthomonas spadix, Rhodothermus marinus, Thermaerobacter marianensis and wherein the Disease Markers (DMs) comprise of Acholeplasma palmae.

5. (canceled)

6. The method according to claim 5, wherein the sequence alignment is performed using one or more of Basic Local Alignment Search Tool (BLAST), BLAST-like alignment tool (BLAT), DIAMOND alignment tool, RAPSearch tool, Burrows-Wheeler Aligner (BWA), Bowtie or through the use of clustering algorithms comprising BLASTCLUST, CLUSTALW, VSEARCH or heuristic techniques of identifying sequence similarity.

7. The method according to claim 1, wherein the plurality of public repositories comprises one or more of NCBI database, Protein Data Bank, KEGG database, PFAM database or EggNOG.

8. (canceled)

9. The method according to claim 1, further comprising calculating the abundance of the sensory protein, comprises:
performing a sequence alignment with the sequences in the created sensory protein sequence database as query against the sequenced metagenomic reads, wherein the hits satisfying a minimum e-value threshold of 0.00001 are considered as correct matches;
computing the cumulative matches of the sequenced metagenomic reads to form a count of sensors for each bacterial strain in the sensory protein sequence database, wherein the count of sensors indicates approximately the potential number of sensory protein coding regions in the genome for that particular bacterial strain for the microbiome sample from which the sequenced metagenomic reads were obtained;
computing the cumulative length of the nucleotide bases for all these hits for each bacterial strain in the sensory protein sequence database to form a covered base length, wherein the covered base length indicates approximately the total length of the potential sensory protein coding regions in the genome for that particular bacterial strain for the microbiome sample from which the sequenced metagenomic reads were obtained;
calculating the sensory protein abundance using one of the following:
calculating ratio of the count of sensors to the total metagenomic size (in Megabases) wherein total metagenomic size (in Megabases) is the size of the sequenced metagenomic reads constituting the microbiome sample, or
calculating the ratio of the covered base length of the particular strain to the total metagenomic size (in Megabases) of the microbiome sample for each available bacterial strain.

10. A system or assessing the risk of prediabetes in a person, the system comprises:
a sample collection module for collecting a microbiome sample from fecal of the person for the assessment of the risk of prediabetes, wherein the microbiome sample comprising microbial cells;
a DNA extractor for extracting DNA from the microbial cells;
a sequencer for sequencing the extracted DNA to get sequenced metagenomic reads;
a database creation module for creating a database of sensory protein sequences of a plurality of organisms, wherein the database of sensory protein sequences comprises information pertaining to the sensory proteins of all fully and partially sequenced bacterial genome obtained from a plurality of public repositories, wherein the database creation module further configured to:
extract a data from the plurality of public repositories,
identify all annotated sensory proteins from the extracted data using a set of keyword searches,
perform a sequence alignment to identify a set of poorly annotated or characterized sensory protein sequences,
filter the results of the sequence alignment based on 95% identity, 95% coverage and an e-value cut-off 1.0*e⁻⁵ (0.00001) to identify a set of additional sensory protein sequences, and
collate the sensory protein sequences and the sequences identified through sequence alignment to create the database of sensory protein sequences;
one or more hardware processors;
a memory in communication with the one or more hardware processors, wherein the one or more first hardware processors are configured to execute programmed instructions stored in the memory, to:
generate sensory protein abundance profiles of case-control samples obtained from publicly available data;
apply a random forest classifier on the generated sensory proteins abundance profiles of case-control samples to generate a classification model, wherein the classification model generation comprises:
applying a Random Forest (RF) approach on the sensory protein abundance profiles of sequenced metagenomic reads,
selecting a random set of sequenced metagenomic reads comprising 90% of the microbiome samples as a training set and rest of the 10% were considered as a test set,
performing 10 replicates on 10-fold cross-validation on the training set to build 100 cross-validation RF models,
capturing an importance of each of the features included in cross-validation models in terms of GINI index,
selecting a predefined number of most ‘important’ features based on GINI index values from each of the 100 cross-validation RF models to obtain a feature sub-set,
ranking each of the features in the feature sub-set, on the basis of the sum of their GINI index values,
obtaining multiple evaluation models by cumulatively adding the next ranked feature in a sub-set of features with the features of the previous ‘evaluation’ model, wherein the first ‘evaluation’ model comprised of the top two features in the feature sub-set,
assessing the performance of all the ‘evaluation’ models on the basis of their added features,
choosing the best performing ‘evaluation’ model based on the assessed performance as the final classification model,
evaluating the performance of the ‘evaluation’ model on the basis of a balancing Score, followed by Matthews correlation coefficient (MCC) and Area under the curve (AUC) scores, and
validating the final classification model on the test set containing rest 10% of the dataset earlier kept aside as the independent test set, wherein the accuracy of training model and the confidence probability of the binary prediction to be ‘case’ or ‘control’ (schizophrenic or healthy) were accounted;
quantify the abundance of a sensory protein from the sequenced metagenomic reads using the database of sensory protein sequences;
assess the risk of the person to be in the prediabetes diseased state using the classification model and the quantified abundance of the sensory protein in the metagenomic sample of the person, wherein the assessment results in the categorization of the person either in a low risk or a high risk of prediabetes diseased state based on a predefined criteria; and
provide a therapeutic construct to the person depending on the risk of the prediabetes.

11. A computer program product comprising a non-transitory computer readable medium having a computer readable program embodied therein, wherein the computer readable program, when executed on a computing device, causes the computing device to:
creating a database of sensory protein sequences of a plurality of organisms, wherein the database of sensory protein sequences comprises information pertaining to the sensory protein of all fully or partially sequenced bacterial genomes obtained from a plurality of public repositories, wherein the creating further comprises:
extracting a data from the plurality of public repositories,
identifying all annotated sensory proteins from the extracted data using a set of keyword searches,
performing a sequence alignment to identify a set of poorly annotated or characterized sensory protein sequences,
filtering the results of the sequence alignment based on 95% identity, 95% coverage and an e-value cut-off 1.0*e⁻⁵ (0.00001) to identify a set of additional sensory protein sequences, and
collating the sensory protein sequences and the sequences identified through sequence alignment to create the database of sensory protein sequences;
generating sensory protein abundance profiles of case-control samples obtained from publicly available data;
applying a random forest classifier on the generated sensory protein abundance profiles of case-control samples to generate a classification model, wherein the classification model generation comprises:
applying a Random Forest (RF) approach on the sensory protein abundance profiles of sequenced metagenomic reads,
selecting a random set of sequenced metagenomic reads comprising 90% of the microbiome samples as a training set and rest of the 10% were considered as a test set,
performing 10 replicates on 10-fold cross-validation on the training set to build 100 cross-validation RF models,
capturing an importance of each of the features included in cross-validation models in terms of GINI index,
selecting a predefined number of most ‘important’ features based on GINI index values from each of the 100 cross-validation RF models to obtain a feature sub-set,
ranking each of the features in the feature sub-set, on the basis of the sum of their GINI index values,
obtaining multiple evaluation models by cumulatively adding the next ranked feature in a sub-set of features with the features of the previous ‘evaluation’ model, wherein the first ‘evaluation’ model comprised of the top two features in the feature sub-set,
assessing the performance of all the ‘evaluation’ models on the basis of their added features,
choosing the best performing ‘evaluation’ model based on the assessed performance as the final classification model,
evaluating the performance of the ‘evaluation’ model on the basis of a balancing Score, followed by Matthews correlation coefficient (MCC) and Area under the curve (AUC) scores, and
validating the final classification model on the test set containing rest 10% of the dataset earlier kept aside as the independent test set, wherein the accuracy of training model and the confidence probability of the binary prediction to be ‘case’ or ‘control’ (schizophrenic or healthy) were accounted;
collecting a microbiome sample from fecal sample of the person for the assessment of the risk of prediabetes, wherein the microbiome sample comprising microbial cells;
extracting DNA from the microbial cells;
sequencing, via a sequencer, using the extracted DNA to get sequenced metagenomic reads;
quantifying the abundance of a sensory protein from the sequenced metagenomic reads using the database of sensory protein sequences;
assessing the risk of the person to be in the prediabetes diseased state using the classification model and the quantified abundance of the sensory protein in the metagenomic sample of the person, wherein the assessment results in the categorization of the person either in a low risk or a high risk of prediabetes diseased state based on a predefined criteria; and
providing a therapeutic construct to the person depending on the risk of the prediabetes.
